(19) United States
(12) Patent Application Publication
(10) Pub. No.: US 2025/0000838 A1
(43) Pub. Date: Jan. 2, 2025

(54) Uses of Inhibitors of YTH Domain Family Proteins in the Management of Cognitive or Developmental Disorders

(71) Applicants: Emory University, Atlanta, GA (US); The University of Chicago, Chicago, IL (US)

(72) Inventors: Peng Jin, Marietta, GA (US); Chuan He, Chicago, IL (US); Zhongyu Zou, Chicago, IL (US)

(21) Appl. No.: 18/701,478

(22) PCT Filed: Oct. 13, 2022
(86) PCT No.: PCT/US2022/046603

§ 371 Date: Apr. 15, 2024

Related U.S. Application Data
(60) Provisional application No. 63/255,039, filed on Oct. 13, 2021.

(51) Int. Cl. A61K 31/343; A61P 25/28; C07K 16/18; C12N 15/113

(52) U.S. Cl. CPC A61K 31/343; A61P 25/28; C07K 16/18; C12N 15/113; C12N 2310/531

(57) ABSTRACT

This disclosure relates to uses of inhibitors of YTH family proteins in the management of fragile X syndrome, fragile X-associated disorders, cognitive disorders, developmental disorders, or central nervous system (CNS) disorders. In certain embodiments, this disclosure relates to treating fragile X syndrome, fragile X-associated disorders, cognitive disorders, developmental disorders, or central nervous system (CNS) disorders by administering an effective amount of an inhibitor of YTHDF1 to a subject in need thereof. In certain embodiments, the YTHDF1 inhibitor is a small molecule inhibitor such as salvianolic acid C, derivatives, prodrugs, esters, or salts thereof.

### CROSS-REFERENCE TO RELATED APPLICATIONS

This application claims the benefit of U.S. Provisional Application No. 63/255,039 filed Oct. 13, 2021. The entirety of this application is hereby incorporated by reference for all purposes.

### STATEMENT REGARDING FEDERALLY SPONSORED RESEARCH OR DEVELOPMENT

This invention was made with government support under HD104458 and HG008935 awarded by the National Institutes of Health. The government has certain rights in the invention.

### BACKGROUND

Fragile X-syndrome (FXS) is caused by a mutation in the fragile X mental retardation 1 (FMR1) gene. This gene encodes a protein called the fragile X mental retardation protein (FMRP), which is required for normal brain development. Thus, FMRP is missing or drastically reduced in FXS, and its absence or reduction in the brain is the cause of the most serious symptoms: cognitive impairment, autistic behaviors, communication and language impairments, seizures, and other features. Thus, there is a need to identify effective therapies.

Cellular ribonucleoprotein (RNP) granules are cellular RNA-protein assemblies which act as hubs of post-transcriptional regulation of RNA metabolism. N6-methyladenosine (m6A) is modification of mRNA in mammalian cells and has implication in epigenetic regulation. YTH family proteins have a domain which specifically bind RNA having the m6A modification and are often referred to as “readers” of the m6A modification. Proteins containing the YT521-B homology (YTH) domain, including YTHDF1-3 and YTHDC1-2 in mammals, are the direct m6A readers possessing a dedicated m6A-binding domain.

Bartley et al. report mammalian FMRP S499 is phosphorylated by CK2 and promotes secondary phosphorylation of FMRP. eNeuro, 2016, 3 (6).

Thomson et al. report cell-type-specific translation profiling reveals a strategy for treating fragile X syndrome. Neuron, 2017, 95, 550-563, e555.

Shi et al. report m6A facilitates hippocampus-dependent learning and memory through YTHDF1. Nature, 2018, 563, 249

Greenblatt et al. report fragile X mental retardation 1 gene enhances the translation of large autism-related proteins. Science, 2018, 361, 709-712.

Tsang et al. report phospho-regulated fragile X mental retardation protein (FMRP) phase separation models activity-dependent translation through bidirectional control of mRNA granule formation. PNSA, 2019, 116, 4218-4227.

Kim et al. report phospho-dependent phase separation of FMRP and CAPRINI recapitulates regulation of translation and deadenylation. Science, 2019, 365, 825-829.

Zhang et al. report genetic analyses support the contribution of mRNA N6-methyladenosine (m6A) modification to human disease heritability. Nat Genetics, 2020, 52, 939-949.

Zaccara et al. report a unified model for the function of YTHDF proteins in regulating m6A-modified mRNA. Cell, 2020, 181, 1582-1595.

Kang et al., report a human forebrain organoid model of fragile X syndrome exhibits altered neurogenesis. Nat Neuroscience, 2021, 24:1377-1391.

Worpenberg et al. report YTHDF is a N6-methyladenosine reader that modulates Fmr1 target mRNA selection and restricts axonal growth in drosophila. EMBO J., 2021, 40, e104975.

References cited herein are not an admission of prior art.

### SUMMARY

This disclosure relates to uses of inhibitors of YTH family proteins in the management of fragile X syndrome, fragile X-associated disorders, cognitive disorders, developmental disorders, or central nervous system (CNS) disorders. In certain embodiments, this disclosure relates to treating fragile X syndrome, fragile X-associated disorders, cognitive disorders, developmental disorders, or central nervous system (CNS) disorders by administering an inhibitor of YTHDF1 to a subject in need thereof. In certain embodiments, the YTHDF1 inhibitor is a small molecule inhibitor such as salvianolic acid C, derivatives, prodrugs, esters, or salts thereof.

In certain embodiments, this disclosure relates to uses of chemical entities for treating fragile X syndrome, developmental disorders, and/or cognitive disorders. This disclosure features chemical entities (e.g., small molecule inhibitors, antisense nucleic acids, peptides, recombinant vectors, CRISPR-sgRNAs, small hairpin RNAs (shRNAs), microRNAs (miRNAs), small interfering RNAs (siRNAs), or combinations thereof) that inhibit YTH domain-containing family proteins (YTHs)), YTF domain family member 1 (YTHDF1), YTF domain family member 2 (YTHDF2), and/or YTF domain family member 3 (YTHDF3).

### BRIEF DESCRIPTION OF THE SEVERAL VIEWS OF THE DRAWINGS

FIG. 1A illustrates the chemical structure of salvianolic acid C (SAC) with the chemical name of (E)-3-(3,4-dihydroxyphenyl)-2-((3-(2-(3,4-dihydroxyphenyl)-7-hydroxybenzofuran-4-yl)acryloyl)oxy)propanoic acid.

FIG. 1B shows inhibition data of SAC against YTHDF1 measured by AlphaScreen™ experiments and a calculated IC₅₀ value.

FIG. 2A shows data indicating inhibition of YTHDF1 rescues the reduced neural progenitor cell (NPC) proliferation in FXS organoids. Quantification data obtained from images on the proportion of Ki67+ and SOX2+ neuronal progenitor cells in either control or FXS forebrain organoids under various treatment conditions at day 56. Data are presented as mean #S.E.M. (n=10 sections from at least from 3 organoids each condition.

FIG. 2B shows quantification data from images indicating inhibition of YTHDF1 delays hastened cell cycle exit of cells in FXS organoids. Control and FXS forebrain organoids were treated with SAC (20 μM) or vehicle (DMSO) from day 49 to day 56 for a week and were labeled with EdU (10 μM) for 24 hours. Ki67− and EdU+ cells in either control or FXS forebrain organoids under various treatment conditions after 24 hours. EdU exposure at day 56.

FIG. 2C shows data indicating YTHDF1 inhibition rescues deficit of altered neuronal differentiation in FXS forebrain organoids. Shown is quantification data from images of PAX6+ or CTIP2+ cells in either control or FXS forebrain organoids under various treatment conditions at day 56.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

“Embodiments” are examples of this disclosure which are not necessarily limited to these examples. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to the idea of excluding certain prior art element(s) as an inventive feature of a claim, but which may contain additional composition components or method steps, etc., that do not materially affect the basic and novel characteristic(s) of the compositions or methods, compared to those of the corresponding compositions or methods disclosed herein.

“Subject” refers to any animal, preferably a human patient, livestock, rodent, monkey, or domestic pet.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

### Inhibitors of YTH Family Proteins

In certain embodiments, this disclosure relates to inhibitors of YTH family proteins. In certain embodiments, the inhibitors of YTH family proteins may be any variety of chemical entities (e.g., small molecule inhibitors, antisense nucleic acids, peptides, recombinant vectors, CRISPR-sgRNAs, small hairpin RNAs (shRNAs), microRNAs (miRNAs), small interfering RNAs (siRNAs), or combinations thereof) that inhibit YTH domain-containing family proteins (YTHs)), YTF domain family member 1 (YTHDF1), YTF domain family member 2 (YTHDF2), and/or YTF domain family member 3 (YTHDF3). In certain embodiments, the inhibitor is salvianolic acid C (SAC) with the chemical name of (E)-3-(3,4-dihydroxyphenyl)-2-((3-(2-(3,4-dihydroxyphenyl)-7-hydroxybenzofuran-4-yl)acryloyl)oxy)propanoic acid, derivatives, prodrugs, esters, or salts thereof.

As used herein, the term “derivative” refers to a structurally similar compound that retains sufficient functional attributes of the identified analogue. The derivative may be structurally similar because it is lacking one or more atoms, e.g., replacing an amino group, hydroxyl, or thiol group with a hydrogen, substituted, a salt, in different hydration/oxidation states, or because one or more atoms within the molecule are switched, such as, but not limited to, replacing an oxygen atom with a sulfur atom, or replacing an amino group with a hydroxyl group. The derivative may be a prodrug, conjugated to a lipid, polyethylene glycol, saccharide, polysaccharide. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

For example, in certain embodiments, the derivative of salvianolic acid C (SAC) is a compound having the following formula:

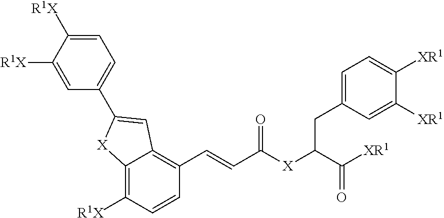

or salt or thereof, wherein each R¹ is individually and independently at each occurrence selected from hydrogen, alkyl, or acyl; and each X is individually and independently at each occurrence selected from O, S, CH2, NH, or Nalkyl.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl. The substituents may further optionally be substituted.

The term “prodrug” refers to an agent that is converted into a biologically active form in vivo. Prodrugs are often useful because, in some situations, they may be easier to administer than the parent compound. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto (thiol) group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol or acetamide, formamide and benzamide derivatives of an amine functional group in the active compound and the like.

In certain embodiments, YTH domain inhibitors are compounds disclosed in WO2021076617, hereby incorporated by reference in its entirety, such as those with the following formula:

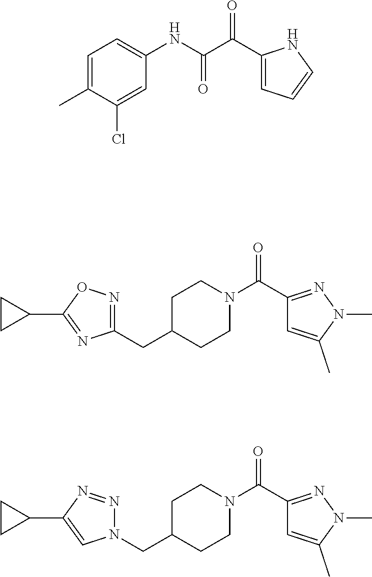 [SMILES: Cc1ccc(NC(=O)C(=O)c2ccc[nH]2)cc1Cl ; Cc1cc(C(=O)N2CCC(Cc3noc(C4CC4)n3)CC2)nn1C ; Cc1cc(C(=O)N2CCC(Cn3cc(C4CC4)nn3)CC2)nn1C]

derivatives, esters, or prodrugs thereof.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs, and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

An “alkoxy” is an alkyl attached to the remainder of the molecule via an oxygen linker (—O—).

The term “acyl” means, unless otherwise stated, —C(O)R where R is a substituted or unsubstituted alkyl.

Certain compounds of the present disclosure possess asymmetric carbon atoms (optical or chiral centers) or double bonds which may be enantiomers, racemates, diastereomers, tautomers, geometric isomers, stereoisomers forms. Compounds may be defined, in terms of absolute stereochemistry, e.g., as (R)- or (S)- or, as (D)- or (L)- for amino acids. Individual isomers are encompassed within the scope of the present disclosure. The compounds of the present disclosure do not include those that are known in art to be too unstable to synthesize and/or isolate. The present disclosure is meant to include compounds as mixtures of isomers or in racemic and optically pure forms. Optically active (R)- and (S)-, or (D)- and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, phosphoric, sulfuric, hydriodic, or phosphorous acids and the like, as well as the salts derived from relatively nontoxic organic acids like acetic, propionic, maleic, malonic, benzoic, succinic, suberic, fumaric, lactic, mandelic, phthalic, benzenesulfonic, citric, tartaric, oxalic, methanesulfonic, and the like. Also included are salts of amino acids such as arginate and the like. Certain specific compounds of the present disclosure contain both basic and acidic functionalities that allow the compounds to be converted into either base or acid addition salts.

In certain contexts, an “antibody” refers to a protein-based molecule that is naturally produced by animals in response to the presence of a protein or other molecule that is not recognized by the animal's immune system to be a “self” molecule i.e., recognized by the animal to be a foreign molecule. The immune system of the animal will create an antibody to specifically bind the antigen/foreign molecule, and thereby targeting the antigen for degradation, or any organism attached to the antigen. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody.

Thus, the term “antibody” is intended to include natural antibodies, monoclonal antibody, or non-naturally produced synthetic antibodies, such as specific binding single chain antibodies, bispecific antibodies, or fragments thereof. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

From a structural standpoint, an antibody is a combination of proteins: two heavy chain proteins and two light chain proteins. The heavy chains are longer than the light chains. The two heavy chains typically have the same amino acid sequence. Similarly, the two light chains typically have the same amino acid sequence. Each of the heavy and light chains contain a variable segment that contains amino acid sequences which participate in binding to the antigen. The variable segments of the heavy chain do not have the same amino acid sequences as the light chains. The variable segments are often referred to as the antigen binding domains. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

The term “antibody fragment” refers to a peptide or polypeptide which comprises less than a complete, intact antibody. Complete antibodies comprise two functionally independent parts or fragments: an antigen binding fragment known as “Fab,” and a carboxy terminal crystallizable fragment known as the “Fc” fragment. The Fab fragment includes the first constant domain from both the heavy and light chain (CH1 and CL1) together with the variable regions from both the heavy and light chains that bind the specific antigen. Each of the heavy and light chain variable regions includes three complementarity determining regions (CDRs) and framework amino acid residues which separate the individual CDRs. The Fc region comprises the second and third heavy chain constant regions (CH2 and CH3) and is involved in effector functions such as complement activation. In some antibodies, the Fc and Fab regions are separated by an antibody “hinge region,” and depending on how the full-length antibody is proteolytically cleaved, the hinge region may be associated with either the Fab or Fc fragment.

In certain embodiments, this disclosure relates to a pharmaceutical composition including an inhibitor described herein and a pharmaceutically acceptable excipient. Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethyl cellulose, hydroxyethyl cellulose, polyvinyl pyrrolidine, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like.

Pharmaceutical compositions include the formulation of an inhibitor disclosed herein with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration. Buffered saline solutions optionally comprising a saccharide or polysaccharide can be used for liquid dosage forms suitable for intravenous administration.

### Methods of Use

In certain embodiments, this disclosure relates to managing fragile X syndrome, fragile X-associated disorders, cognitive disorders, developmental disorders, or central nervous system (CNS) disorders by administering an inhibitor of YTHDF1 to a subject in need thereof. In certain embodiments, the YTHDF1 inhibitor is a small molecule inhibitor such as E)-3-(3,4-dihydroxyphenyl)-2-((3-(2-(3,4-dihydroxyphenyl)-7-hydroxybenzofuran-4-yl)acryloyl)oxy)propanoic acid [salvianolic acid C], derivatives, prodrugs, esters, or salts thereof.

In certain embodiments, this disclosure relates to uses of chemical entities for treating or preventing fragile X syndrome, fragile X-associated disorders, cognitive disorders, developmental disorders, or central nervous system (CNS) disorders. This disclosure features chemical entities (e.g., small molecule inhibitors, antisense nucleic acids, peptides, recombinant vectors, CRISPR-sgRNAs, small hairpin RNAs (shRNAs), microRNAs (miRNAs), small interfering RNAs (siRNAs), or combinations thereof) that inhibit YTH domain-containing family proteins (YTHs)), YTF domain family member 1 (YTHDF1), YTF domain family member 2 (YTHDF2), and/or YTF domain family member 3 (YTHDF3).

In certain embodiments, this disclosure relates to methods of treating or preventing fragile X syndrome, fragile X-associated disorders, cognitive disorders, developmental disorders, or central nervous system (CNS) disorders comprising administering an effective amount of a YTHDF inhibitor to a subject in need thereof. In certain embodiments, the YTHDF inhibitor is a selective small-molecule inhibitor of YTHDF1. In certain embodiments, the YTHDF inhibitor is (E)-3-(3,4-dihydroxyphenyl)-2-((3-(2-(3,4-dihydroxyphenyl)-7-hydroxybenzofuran-4-yl)acryloyl)oxy)propanoic acid [salvianolic acid C], derivatives, prodrugs, esters, or salts thereof.

In certain embodiments, the fragile X-associated disorder is fragile X syndrome (FXS), fragile X-associated primary ovarian insufficiency (FXPOI) and fragile X-associated tremor/ataxia syndrome (FXTAS).

In certain embodiments, the YTHDF inhibitor is an antibody that specifically binds with an epitope on a YTHDF1 polypeptide.

In certain embodiments, YTHDF inhibitor is interfering shRNA or vector encoding interfering shRNA that targets YTHF1 mRNA or an antisense nucleobase polymer that targets YTHF1 mRNA.

Fragile X syndrome is typically diagnosed using PCR to identify genetic loci with polymorphisms. An example of a locus exhibiting a medically relevant polymorphism is the 5′ untranslated region (UTR) of the human FMR1 gene on the X chromosome. Conventional systems use CGG-repeat primed (TRP)—PCR resolved by CE electropherogram to identify alleles of a locus containing large CGG repeat expansion. Normal individuals typically have 5-44 CGG repeats in this locus. In contrast, alleles of this locus containing large CGG repeat expansion (>200 repeats, full mutation alleles) disrupts FMR1 gene expression and causes FXS. Moreover, individuals with a premutation (PM) allele (50-200 repeats) are at risk of developing late-onset neurodegenerative disease fragile X-associated tremor/ataxia syndrome (FXTAS) or fragile X-associated primary ovarian insufficiency (FXPOI). Female PM allele carriers are at risk of transmitting full mutation alleles to their offspring. This risk depends on the size of CGG repeats, measured by a Fragile X PCR assay, and the number of AGG interruptions among the CGG repeats.

In certain embodiments, this disclosure relates to methods of treating a developmental disorder comprising administering an effective amount of a YTHDF inhibitor to a subject in need thereof. In certain embodiments, the YTHDF inhibitor is a selective small-molecule inhibitor of YTHDF1. In certain embodiments, the YTHDF inhibitor is (E)-3-(3,4-dihydroxyphenyl)-2-((3-(2-(3,4-dihydroxyphenyl)-7-hydroxybenzofuran-4-yl)acryloyl)oxy)propanoic acid [salvianolic acid C], derivatives, prodrugs, esters, or salts thereof.

In certain embodiments, the YTHDF inhibitor is an antibody with an epitope on a YTHDF1 polypeptide.

In certain embodiments, the YTHDF inhibitor is interfering shRNA or vector encoding interfering shRNA that targets YTHF1 mRNA or an antisense nucleobase polymer that targets YTHF1 mRNA.

In certain embodiments, the developmental disorder is a poor motor coordination, prominent ears, a long face, flat feet, hyperextensible finger joints, double-jointed thumbs, an infant with weak sucking and/or frequent regurgitation.

In certain embodiments, this disclosure relates to methods of treating a cognitive disorder or central nervous system (CNS) disorder comprising administering an effective amount of a YTHDF inhibitor to a subject in need thereof.

In certain embodiments, the YTHDF inhibitor is a selective small-molecule inhibitor of YTHDF1. In certain embodiments, the YTHDF inhibitor is (E)-3-(3,4-dihydroxyphenyl)-2-((3-(2-(3,4-dihydroxyphenyl)-7-hydroxybenzofuran-4-yl)acryloyl)oxy)propanoic acid [salvianolic acid C], derivatives, prodrugs, esters, or salts thereof.

In certain embodiments, the YTHDF inhibitor is an antibody with an epitope on a YTHDF1 polypeptide.

In certain embodiments, YTHDF inhibitor is interfering shRNA or vector encoding interfering shRNA that targets YTHF1 mRNA or an antisense nucleobase polymer that targets YTHF1 mRNA.

In certain embodiments, the cognitive disorder is an autism spectrum disorder, attention-deficit/hyperactivity disorder (ADHD), speech impediment, hyperactivity, seizure, and/or a hypotonic state.

In certain embodiments, this disclosure relates to methods of treating cognitive impairment associated with central nervous system (CNS) disorders in a subject in need or at risk thereof, including, without limitation, subjects having, diagnosed with, or at risk for age-related cognitive impairment, Mild Cognitive Impairment (MCI), amnestic MCI (aMCI), Age-Associated Memory Impairment (AAMI), Age Related Cognitive Decline (ARCD), dementia, Alzheimer's Disease (AD), prodromal AD, post-traumatic stress disorder (PTSD), schizophrenia, bipolar disorder, amyotrophic lateral sclerosis (ALS), cancer-therapy-related cognitive impairment, mental retardation, Parkinson's disease (PD), autism spectrum disorders, Rett syndrome, compulsive behavior, and substance addiction.

In some embodiments, inhibitors of this disclosure may be use in treating cancer or brain cancers (including brain tumors, e.g., medulloblastomas) comprising administering an effective amount of an inhibitor disclosed herein. In some embodiments, inhibitors of this disclosure may be use in treating cognitive impairment associated with brain cancers (including brain tumors, e.g., medulloblastomas). In some embodiments, inhibitors of this disclosure may be used in treating intestinal cancer. In some embodiments, inhibitors of this disclosure may be used in treating colorectal cancer. In some embodiments, inhibitors of this disclosure may be used in treating gastric cancer. In some embodiments, inhibitors of this disclosure may be used in treating lung cancer. In some embodiments, inhibitors of this disclosure may be used in treating non-small cell lung cancer (NSCLC). In some embodiments, inhibitors of this disclosure may be used in treating or preventing drug resistance or metastasis. In some embodiments, inhibitors of this disclosure may be used in treating ovarian cancer. In some embodiments, inhibitors of this disclosure may be used in treating hepatocellular carcinoma. In some embodiments, inhibitors of this disclosure may be used in treating pancreatic cancer. In some embodiments, inhibitors of this disclosure may be used in treating bladder cancer. In some embodiments, inhibitors of this disclosure may be used in treating prostate cancer. In some embodiments, inhibitors of this disclosure may be used in treating Merkel cell carcinoma. In some embodiments, inhibitors of this disclosure may be used in treating a hematological cancer such as acute myeloid leukemia. In some embodiments, inhibitors of this disclosure may be used in treating ocular melanoma. In some embodiments, inhibitors of this disclosure may be used in treating brain cancer or glioblastoma. In some embodiments, inhibitors of this disclosure may be used in treating endometrial cancer or carcinoma. In some embodiments, inhibitors of this disclosure may be used in treating nasopharyngeal cancer or carcinoma.

“Cognitive function” or “cognitive status” refers to any higher order intellectual brain process or brain state, respectively, involved in learning and/or memory including, but not limited to, attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, expressing an interest in one's surroundings and self-care, speed of processing, reasoning and problem solving and social cognition.

“Treating cognitive impairment” refers to taking steps to improve cognitive function in a subject with cognitive impairment so that the subject's performance in one or more cognitive tests is improved to any detectable degree or is prevented from further decline. Preferably, that subject's cognitive function, after treatment of cognitive impairment, more closely resembles the function of a normal, unimpaired subject. Treatment of cognitive impairment in humans may improve cognitive function to any detectable degree but is preferably improved sufficiently to allow the impaired subject to carry out daily activities of normal life at the same level of proficiency as a normal, unimpaired subject. In some cases, “treating cognitive impairment” refers to taking steps to improve cognitive function in a subject with cognitive impairment so that the subject's performance in one or more cognitive tests is improved to any detectable degree or is prevented from further decline. Preferably, that subject's cognitive function, after treatment of cognitive impairment, more closely resembles the function of a normal, unimpaired subject. In some cases, “treating cognitive impairment” in a subject affecting by age-related cognitive impairment refers to takings steps to improve cognitive function in the subject so that the subject's cognitive function, after treatment of cognitive impairment, more closely resembles the function of an age-matched normal, unimpaired subject, or the function of a young adult subject.

In animal model systems, cognitive function may be measured in various conventional ways known in the art, including using a Morris Water Maze (MWM), Barnes circular maze, elevated radial arm maze, T maze or any other mazes in which the animals use spatial information. Cognitive function can be assessed by reversal learning, extradimensional set shifting, conditional discrimination learning and assessments of reward expectancy. Other tests known in the art may also be used to assess cognitive function, such as novel object recognition and odor recognition tasks. Preventing a cognitive disorder may be characterized as “preserving” cognitive function as it results in normal or impaired cognitive function such that it does not decline or does not fall below that observed in the subject upon first presentation, diagnosis, or delays such decline.

Mental retardation is a generalized disorder characterized by significantly impaired cognitive function and deficits in adaptive behaviors. Mental retardation is often defined as an Intelligence Quotient (IQ) score of less than 70. The dysfunction in neuronal communication is also considered one of the underlying causes for mental retardation (Myrrhe van Spronsen and Casper C. Hoogenraad, Curr. Neurol. Neurosci. Rep. 2010, 10, 207-214).

In some instances, mental retardation includes, but are not limited to, Down syndrome, velocardiofacial syndrome, fetal alcohol syndrome, Fragile X syndrome, Klinefelter's syndrome, neurofibromatosis, congenital hypothyroidism, Williams syndrome, phenylketonuria (PKU), Smith-Lemli-Opitz syndrome, Prader-Willi syndrome, Phelan-McDermid syndrome, Mowat-Wilson syndrome, ciliopathy, Lowe syndrome, and X-linked mental retardation. Down syndrome is a disorder that includes a combination of birth defects, including some degree of mental retardation, characteristic facial features and, often, heart defects, increased infections, problems with vision and hearing, and other health problems. Fragile X syndrome is a prevalent form of inherited mental retardation characterized by developmental delay, hyperactivity, attention deficit disorder, and autistic-like behavior.

“Cognitive impairment” refers to cognitive function in subjects that is not as robust as that expected in a normal, unimpaired subject. In some cases, cognitive function is reduced by about 5%, about 10%, about 30%, or more, compared to cognitive function expected in a normal, unimpaired subject. In some cases, “cognitive impairment” in subjects affected by aged-related cognitive impairment refers to cognitive function in subjects that is not as robust as that expected in an aged-matched normal, unimpaired subject, or the function of a young adult subject (i.e. subjects with mean scores for a given age in a cognitive test).

“Age-related cognitive impairment” refers to cognitive impairment in aged subjects, wherein their cognitive function is not as robust as that expected in an age-matched normal subject or as that expected in young adult subjects. In some cases, cognitive function is reduced by about 5%, about 10%, about 30%, or more, compared to cognitive function expected in an age-matched normal subject. In some cases, cognitive function is as expected in an age-matched normal subject, but reduced by about 5%, about 10%, about 30%, about 50% or more, compared to cognitive function expected in a young adult subject. Age-related impaired cognitive function may be associated with Mild Cognitive Impairment (MCI) (including amnestic MCI and non-amnestic MCI), Age-Associated Memory Impairment (AAMI), and Age-related Cognitive Decline (ARCD).

“Cognitive impairment” associated with AD or related to AD or in AD refers to cognitive function in subjects that is not as robust as that expected in subjects who have not been diagnosed AD using conventional methodologies and standards.

“Mild Cognitive Impairment” or “MCI” refers to a condition characterized by isolated memory impairment unaccompanied other cognitive abnormalities and relatively normal functional abilities. One set of criteria for a clinical characterization of MCI specifies the following characteristics: (1) memory complaint (as reported by patient, informant, or physician), (2) normal activities of daily living (ADLs), (3) normal global cognitive function, (4) abnormal memory for age (defined as scoring more than 1.5 standard deviations below the mean for a given age), and (5) absence of indicators of dementia (as defined by DSM-IV guidelines). Petersen et al., Srch. Neurol. 56:303-308 (1999); Petersen, “Mild cognitive impairment: Aging to Alzheimer's Disease.” Oxford University Press, N.Y. (2003). The cognitive deficit in subjects with MCI may involve any cognition area or mental process including memory, language, association, attention, perception, problem solving, executive function and visuospatial skills. See, e.g., Winbald et al., J. Intern. Med. 256:240-240, 2004; Meguro, Acta. Neurol. Taiwan. 15:55-57, 2008; Ellison et al., CNS Spectr. 13:66-72, 2008, Petersen, Semin. Neurol. 27:22-31, 2007. MCI is further subdivided into amnestic MCI (aMCI) and non-amnestic MCI, characterized by the impairment (or lack thereof) of memory in particular.

“Age-Associate Memory Impairment (AAMI)” refers to a decline in memory due to aging. A patient may be considered to have AAMI if he or she is at least 50 years old and meets all of the following criteria: a) The patient has noticed a decline in memory performance, b) The patient performs worse on a standard test of memory compared to young adults, c) All other obvious causes of memory decline, except normal aging, have been ruled out (in other words, the memory decline cannot be attributed to other causes such as a recent heart attack or head injury, depression, adverse reactions to medication, Alzheimer's disease, etc.).

“Age-Related Cognitive Decline (ARCD)” refers to declines in memory and cognitive abilities that are a normal consequence of aging in humans (e.g., Craik & Salthouse, 1992). This is also true in virtually all mammalian species. Age-Associated Memory Impairment refers to older persons with objective memory declines relative to their younger years, but cognitive functioning that is normal relative to their age peers (Crook et al., 1986). Age-Consistent Memory Decline is a less pejorative label which emphasizes that these are normal developmental changes (Crook, 1993; Larrabee, 1996), are not pathophysiological (Smith et al., 1991), and rarely progress to overt dementia (Youngjohn & Crook, 1993). The DSM-IV (1994) has codified the diagnostic classification of ARCD.

“Dementia” refers to a condition characterized by severe cognitive deficit that interferes in normal activities of daily living. Subjects with dementia also display other symptoms such as impaired judgment, changes in personality, disorientation, confusion, behavior changes, trouble speaking, and motor deficits. There are different types of dementias, such as Alzheimer's disease (AD), vascular dementia, dementia with Lewy bodies, and frontotemporal dementia.

Alzheimer's disease (AD) is characterized by memory deficits in its early phase. Later symptoms include impaired judgment, disorientation, confusion, behavior changes, trouble speaking, and motor deficits. Histologically, AD is characterized by beta-amyloid plaques and tangles of protein tau. Dementia with Lewy bodies is characterized by abnormal deposits of alpha-synuclein that form inside neurons in the brain. Cognitive impairment may be similar to AD, including impairments in memory and judgment and behavior changes.

Frontotemporal dementia is characterized by gliosis, neuronal loss, superficial spongiform degeneration in the frontal cortex and/or anterior temporal lobes, and Picks' bodies. Symptoms include changes in personality and behavior, including a decline in social skills and language expression/comprehension.

“Post-traumatic stress disorder (PTSD)” refers to an anxiety disorder characterized by an immediate or delayed response to a catastrophic event, characterized by re-experiencing the trauma, psychic numbing or avoidance of stimuli associated with the trauma, and increased arousal. Re-experiencing phenomena include intrusive memories, flashbacks, nightmares, and psychological or physiological distress in response to trauma reminders. Such responses produce anxiety and can have significant impact, both chronic and acute, on a patient's quality of life and physical and emotional health. PTSD is also associated with impaired cognitive performance, and older individuals with PTSD have greater decline in cognitive performance relative to control patients.

“Schizophrenia” refers to a chronic debilitating disorder, characterized by a spectrum of psychopathology, including positive symptoms such as aberrant or distorted mental representations (e.g., hallucinations, delusions), negative symptoms characterized by diminution of motivation and adaptive goal-directed action (e.g., anhedonia, affective flattening, avolition), and cognitive impairment. While abnormalities in the brain are proposed to underlie the full spectrum of psychopathology in schizophrenia, currently available antipsychotics are largely ineffective in treating cognitive impairments in patients.

“Bipolar disorder” or “BP” or “manic depressive disorder” or “manic depressive illness” refers to a chronic psychological/mood disorder which can be characterized by significant mood changes including periods of depression and euphoric manic periods. BP may be diagnosed by a skilled physician based on personal and medical history, interview consultation and physical examinations. The term “mania” or “manic periods” or other variants refers to periods where an individual exhibits some or all of the following characteristics: racing thoughts, rapid speech, elevated levels of activity and agitation as well as an inflated sense of self-esteem, euphoria, poor judgment, insomnia, impaired concentration, and aggression.

“Amyotrophic lateral sclerosis,” also known as ALS, refers to a progressive, fatal, neurodegenerative disease characterized by a degeneration of motor neurons, the nerve cells in the central nervous system that control voluntary muscle movement. ALS is also characterized by neuronal degeneration in the entorhinal cortex and hippocampus, memory deficits, and neuronal hyperexcitability in different brain areas such as the cortex.

“Cancer-therapy-related cognitive impairment” refers to cognitive impairment that develops in subjects that are treated with cancer therapies such as chemotherapy (e.g., chemobrain) and radiation. Cytotoxicity and other adverse side-effects on the brain of cancer therapies result in cognitive impairment in such functions as memory, learning and attention.

Parkinson's disease (PD) is a neurological disorder characterized by a decrease of voluntary movements. The afflicted patient has reduction of motor activity and slower voluntary movements compared to the normal individual. The patient has characteristic “mask” face, a tendency to hurry while walking, bent over posture and generalized weakness of the muscles. There is a typical “lead-pipe” rigidity of passive movements. Another important feature of the disease is the tremor of the extremities occurring at rest and decreasing during movements.

“Autism,” as used herein, refers to an autism spectrum disorder characterized by a neural development disorder leading to impaired social interaction and communication by restricted and repetitive behavior. “Autism Spectrum Disorder” refers to a group of developmental disabilities that includes: autism; Asperger syndrome; pervasive developmental disorder not otherwise specified (PDD-NOS or atypical autism); Rett syndrome; and childhood disintegrative disorder.

Obsessive compulsive disorder (“OCD”) is a mental condition that is most commonly characterized by intrusive, repetitive unwanted thoughts (obsessions) resulting in compulsive behaviors and mental acts that an individual feels driven to perform (compulsion). Current epidemiological data indicates that OCD is the fourth most common mental disorder in the United States. Some studies suggest the prevalence of OCD is between one and three percent, although the prevalence of clinically recognized OCD is much lower, suggesting that many individuals with the disorder may not be diagnosed. Patients with OCD are often diagnosed by a psychologist, psychiatrist, or psychoanalyst according to the Diagnostic and Statistical Manual of Mental Disorders, 4th edition text revision (DSM-IV-TR) (2000) diagnostic criteria that include characteristics of obsessions and compulsions.

Substance addiction (e.g., drug addiction, alcohol addiction) is a mental disorder. The addiction is not triggered instantaneously upon exposure to substance of abuse. Rather, it involves multiple, complex neural adaptations that develop with different time courses ranging from hours to days to months (Kauer J. A. Nat. Rev. Neurosci. 2007, 8, 844-858). The path to addiction generally begins with the voluntary use of one or more controlled substances, such as narcotics, barbiturates, methamphetamines, alcohol, nicotine, and any of a variety of other such controlled substances. Over time, with extended use of the controlled substance(s), the voluntary ability to abstain from the controlled substance(s) is compromised due to the effects of prolonged use on brain function, and thus on behavior. As such, substance addiction generally is characterized by compulsive substance craving, seeking and use that persist even in the face of negative consequences. The cravings may represent changes in the underlying neurobiology of the patient which likely must be addressed in a meaningful way if recovery is to be obtained. Substance addiction is also characterized in many cases by withdrawal symptoms, which for some substances are life threatening (e.g., alcohol, barbiturates) and in others can result in substantial morbidity (which may include nausea, vomiting, fever, dizziness, and profuse sweating), distress, and decreased ability to obtain recovery. For example, alcoholism, also known as alcohol dependence, is one such substance addiction. Alcoholism is primarily characterized by four symptoms, which include cravings, loss of control, physical dependence, and tolerance. These symptoms also may characterize addictions to other controlled substances.

As used herein, the term “administering” means oral administration, administration as a suppository, topical contact, intravenous, parenteral, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

The term “therapeutically effective amount,” as used herein, refers to that amount of the therapeutic agent sufficient to ameliorate the disorder, as described herein. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Phase Separation into Distinct Ribonucleoprotein (RNP) Granules Dictate Translation Outcome Through YTHDF1

Fragile X syndrome, a genetic disorder linked to autism, is caused by the loss of functional fragile X mental retardation protein (FMRP). FMRP is a selective RNA-binding protein that forms a messenger ribonucleoprotein (mRNP) complex associating with polyribosomes. Evidence suggests that FMRP is involved in the regulation of local protein synthesis at synapses. The loss of FMRP leads to abnormal translation of selective mRNAs, many of which are autism-linked genes.

An FXS human forebrain organoid model has been developed where one can observed the loss of FMRP which leads to dysregulated neurogenesis, neuronal maturation, and neuronal excitability indicating the utility of FXS organoid model for drug screening and testing. Interestingly, the mRNA targets of FMRP are enriched for m6A marks. Biochemical and genetic interactions between FMRP and the m6A modification pathway were observed. N6-methyladenosine (m6A) modification on mammalian mRNA facilitates cellular ribonucleoprotein (RNP) granule formation by recruiting its reader YTHDF proteins, including YTHDF1. The YTHDF1 mediated translation can be modulated by FMRP when unphosphorylated FMRP forms a RNP granule with YTHDF1 to exclude it from ribosome-containing granules. Hyperactive YTHDF1-mediated regulation associated with FMR1 deficiency can be targeted with selective inhibitors of YTHDF1. These inhibitors rescue the neurodevelopmental deficits associated with the loss of FMRP in an FXS organoid model. Thus, YTHDF1 is a therapeutic target for fragile X syndrome and the selective inhibitors of YTHDF1 may be used to treat FXS and other associated conditions.

N6-methyladenosine (m6A) modification on mammalian mRNA facilitates cellular ribonucleoprotein (RNP) granule formation by recruiting its reader YTHDF proteins. Experiments reported herein indicate that YTHDF proteins regulate translation of their mRNA targets by dynamic regulation of RNP granule formation whereby YTHDF1 condenses with the 40S ribosomal subunit to promote translation of its RNA targets. The YTHDF1 mediated translation can be altered by FMRP when unphosphorylated FMRP forms RNP granule with YTHDF1 to hijack it away from ribosome-containing granules. This indicates that hyperactive YTHDF1-mediated translation caused by FMR1 defects can be targeted with a selective inhibitor of YTHDF1 as a method to treat fragile X syndrome (FXS) or other related disorders.

### FMRP is Involved in Depolarization Induced Translation in Neurons

To study the regulatory mechanisms of how YTHDF1 functions in neurons, YTHDF1 activation upon neuronal depolarization was investigated. During mouse neuronal depolarization, YTHDF1 transitions from an “inactivated” state to an “activated” state to promote translation in wild-type neurons. Shi et al. (2018) report Ythdf1 negative knockout in mouse hippocampal neurons caused impaired protein synthesis in stimulated neurons. Cascades of protein phosphorylation are the main molecular pathway through which neurons respond to extracellular stimulation. Thus, experiments were performed to determine whether there was a change in YTHDF1 protein levels or phosphorylation upon stimulation. In mouse hippocampal neurons, a noticeable change in either the YTHDF1 protein level or its phosphorylation was not observed. If YTHDF1 does not undergo noticeable phosphorylation in response to neuronal stimulation, there could exist other RNA binding proteins that respond to protein phosphorylation cascades upon neuronal stimulation. The altered phosphorylation state of this protein could affect the YTHDF1-mediated RNA translation. To identify possible regulators of YTHDF1, genetic variants associated with m6A levels in mRNA transcripts, or m6A quantitative trait loci (m6A-QTLs) were examined. Integrating m6A-QTL results with other molecular QTL data, provided a list of proteins that are potentially involved in m6A-mediated translation. Among these proteins, YTHDF1 exerts an effect on m6A-dependent translation upregulation. Fragile X mental retardation protein (FMRP) was identified as a potential target because it preferentially binds m6A-modified mRNA. Similar to YTHDF1, FMRP preferentially binds the 3′-UTR of its target genes in HEK293T cells. In addition, the center of YTHDF1 binding sites overlapped well with FMRP binding sites.

To assess the regulatory effects of FMRP on YTHDF1-regulated translation, the FMR1 gene was silenced in HEK293T cells and a tethering assay was performed. YTHDF1 posed a slight suppressive effect on its RNA targets while knockdown of FMR1 reversed this effect. As a control, knockdown of YBX3 (which was reported to repress the translation of m6A-tagged transcripts) did not alter the role of YTHDF1 in translation.

To study effects of FMR1 in a more relevant cellular system, a neuron progenitor cell (NPC) differentiation protocol was adopted to produce cultured neurons. Depolarization-stimulated nascent protein synthesis in differentiated neurons using KCl was reproduced. KCl depolarization induced noticeable increases in protein synthesis rate in WT neurons ten minutes post stimulation. The protein synthesis rate returned to the basal level six hours after stimulation, which is consistent with the previous report in Shi et al., 2018. Intriguingly, the elevated nascent protein synthesis was not observed in Fmr1-KO neurons, similar to the observed outcome in the Ythdf1-KO neurons (Shi et al., 2018).

Experiments were performed to determine whether the effect of FMRP on nascent protein synthesis is related to the YTHDF1-dependent mRNA translation. A reporter system was utilized in which the N terminus (amino acid 1-362) of YTHDF1 (YTHDF1-N) is tethered to the 3′ UTR of the firefly luciferase (F-Luc) coding sequence, mimicking the direct binding of YTHDF1 to its target sequences. The YTHDF1-bound F-Luc translation was elevated after KCl depolarization in the wild-type neurons, while no significant change was observed in Fmr1-KO neurons. These data indicate that FMRP is important for stimulating YTHDF1 activation.

### FMRP Phosphorylation Regulates YTHDF1 Phase Separation

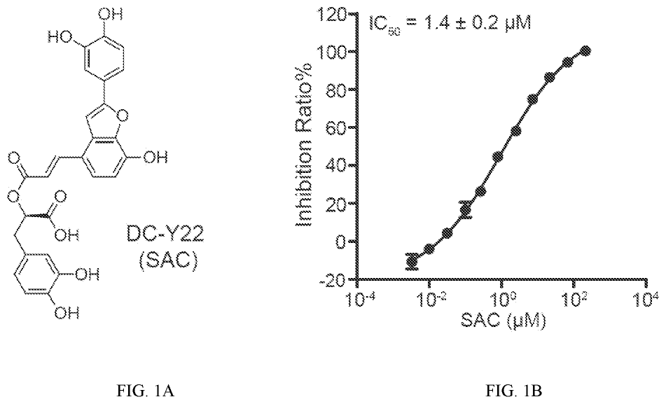

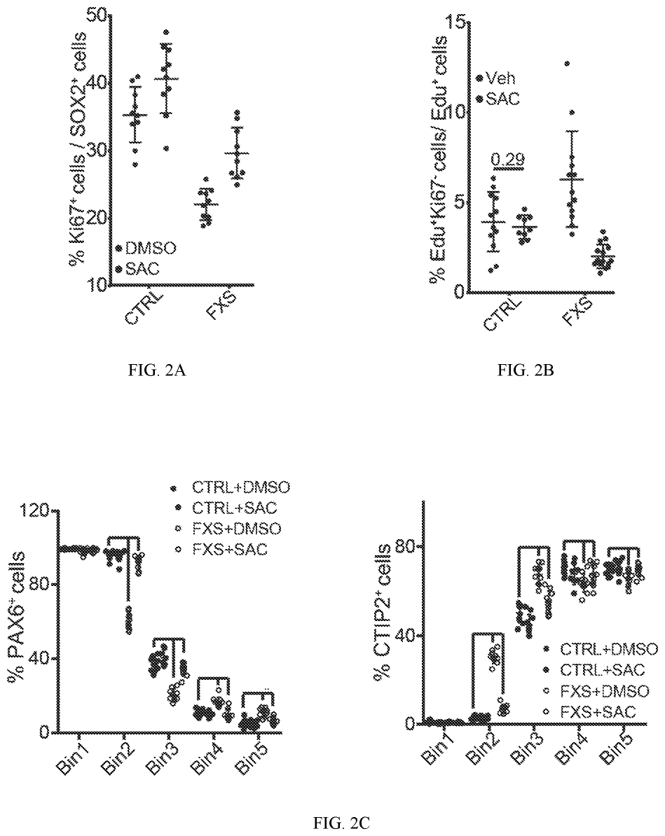

Phosphorylation of FMRP is reported to regulate phase separation and mediate trafficking of mRNA between cellular granules Kim et al., 2019. Phosphorylation of FMRP might act as a functional switch for YTHDF1. Cell lysates from depolarized neurons were collected and probed for FMRP phosphorylation using an antibody against the major form of phosphorylated FMRP (S499). Increased FMRP phosphorylation was observed as early as ten minutes post-stimulation. The phosphorylation level of FMRP reduced to basal level after two to six hours (FIG. 1D). Because this phosphorylation pattern matches that of the YTHDF1-mediated translation upregulation during neuronal depolarization, additional experiments were performed to determine how FMRP phosphorylation regulates YTHDF1 function.

Neurons were treated with inhibitors of FMRP. Casein kinase 2 (CK2) mediates phosphorylation of FMRP at serine 499 (S499) (Bartley et al., 2016) and protein phosphatase 2A (PP2A) catalyzes the dephosphorylation. Silmitasertib (CX-4945) is a potent and selective inhibitor of CK2 (casein kinase 2) and results in loss of p-FMRP while okadaic acid (OA) inhibits PP2A and leads to elevated FMRP phosphorylation. Wild-type differentiated neurons with CX-4945 were treated and probed for nascent protein synthesis. The inhibitor treatment ablated the elevated protein synthesis caused by depolarization using KCl in the ten-minute to two-hour window in these neurons. These data indicate that increased FMRP phosphorylation, upon neuronal stimulation and CK2 activation, is important for YTHDF1-dependent translation.

Because the FMRP phosphorylation state affects its propensity to form phase separated droplets and subsequently alters granule formation and RNA translation, experiments were performed to determine whether this increased phosphorylation during neuronal stimulation could affect YTHDF1 condensation. High-resolution imaging of YTHDF1 protein was performed on immunolabeled neuron samples using direct stochastic optical reconstruction microscopy (dSTORM). Three stages as time points were selected for fixation to visualize YTHDF1 aggregation: no stimulation, ten minutes post-stimulation and two hours post-stimulation. Analysis of dSTORM datasets revealed an increase of cluster size after stimulation in WT neurons, with neurons fixed 10 min post-stimulation exhibiting a higher population of larger clusters. This increase of YTHDF1 clustering coincides with upregulated translation and could be a molecular signature of YTHDF1 activation. In Fmr1-KO neurons where YTHDF1 could be constitutionally activated, this increase was not observed. The basal YTHDF1 cluster size in Fmr1-KO neurons is higher compared to WT neurons, corroborating the notion that increased YTHDF1 clustering is a sign of YTHDF1 activation. No significant re-distribution of YTHDF1 protein in dendrite versus soma was observed during neuronal stimulation.

YTHDF1 Condensate Interacts with the Small Ribosomal Subunit to Promote Translation

After observing the co-occurrence of YTHDF1 clustering and increased translation promoted by YTHDF1, experiments were performed to establish their functional relationship and explore the underlying molecular mechanism. Because the N terminus of YTHDF1 (amino acid 1-362) was previously shown to be sufficient to promote RNA translation, it was reasoned that phase separation of YTHDF1 might contribute to the increased ribosome association and translation promotion. The recombinant N terminus of YTHDF1 expressed in E. coli was purified, and it was confirmed that this portion of YTHDF1 alone is sufficient to form phase separated droplets in vitro under physiological conditions.

Experiments were performed to determine whether YTHDF1 phase separation is sufficient to elevate translation of its target mRNAs in an in vitro translation system with rabbit reticulocyte lysate (RRL). Capped reporter mRNA bearing coding sequences of firefly luciferase (F-Luc-2BoxB-2MS2) and Renilla luciferase (R-Luc) were purified from in vitro transcription reactions and used as probes for the in vitro translation assay. The N terminal domain of YTHDF1 fused with MS2 (YTHDF1N-MS2) was used as well as an HA-MS2 fusion control. The presence of YTHDF1-N tethering leads to increased translation of the reporter mRNA compared with the HA-MS2 control. Inhibition of phase separation using 1,6-hexanediol (HDO), a known LLPS inhibitor, noticeably reduced the translation of the F-Luc reporter with YTHDF1N-MS2 but did not do so for the HA-MS2 control. These data suggest that LLPS is important for YTHDF1-mediated translation promotion in vitro.

Experiments were performed to determine whether translation upregulation through YTHDF1 phase separation also occurs inside cells. HEK293T cells were selected because they have a neuronal crest origin during development and expressed several neuron-specific genes. Initial experiments tested whether YTHDF1 can form LLPS droplets by inducing protein condensation in cell lysates with an RNase A/T1 treatment. The insoluble fraction was separated via centrifugation with a protocol to separate RNA granules in cultured cells. The cell lysate solution became turbid after RNase A/T1 treatment for ten minutes at 37° C., indicating successful induction of LLPS droplet formation. Blotting of separated cellular fractions showed YTHDF1 enrichment in the RNP granule (RG) fraction after RNase treatment. This enrichment of YTHDF1 in insoluble fraction suggests that RNase treatment could induce YTHDF1 phase separation. Other translation-related proteins were blotted, and it was observed that the 40S ribosomal subunit marker ribosomal protein S6 (RPS6) also displayed similar RNase-treatment-dependent enrichment in the insoluble fraction. FMRP does not respond to RNase treatment and remained largely in the soluble fractions. Interestingly, YTHDF2 showed a different pattern in this process. YTHDF2 solubility is largely unaffected by RNase treatment and seems to form insoluble granules in either case (with or without RNase treatment). A consistent trend was also observed in HeLa cells, suggesting that this is likely a conserved feature of YTHDF proteins in different mammalian cell lines. A panel of proteins that form condensates in response to RNase-induced phase separation was also observed. RNase-IP is modified from a protein co-immunoprecipitation (co-IP) procedure that includes RNase treatment steps performed at 37° C. to avoid disturbing temperature and salt-sensitive LLPS-enabled granule assembly. An RNase-IP from HEK293T cell lysate was performed using an antibody specific to YTHDF1. RPS6 was detected in the YTHDF1 IP fractions. Upon RNase treatment, significantly more RPS6 protein was detected in the IP fractions. An increased association between YTHDF1 and the small ribosomal subunit was therefore observed upon phase separation.

To confirm whether the increased interaction between the 40S ribosomal subunit and YTHDF1 protein is dependent on phase separation inside cells. Polysome profiling was performed in HEK293T cells treated with 5% 1,6-hexanediol, which induced global translation arrest. eIF3b and YTHDF1 showed a similar distribution pattern in the absence of 1,6-hexanediol. Upon 1,6-hexanediol treatment, polysomes disassembled and YTHDF1 footprint in the monosome fraction was mostly diminished. Cells treated with 200 mM KCl were also included as these conditions inhibit LLPS. Polysome disassembly was observed and YTHDF1 retention in the 40S fraction was also lowered. These data collectively indicate that the association of YTHDF1 with the 40S ribosomal subunit is dependent on phase separation, and that YTHDF1 phase separation could facilitate interaction with the small ribosomal subunit for translation promotion of its target mRNA.

FMRP Inhibits YTHDF1 Phase Separation with the Small Ribosomal Subunit

YTHDF1 was first described to promote translation of its binding transcripts in HeLa cells but later results suggest that it does not significantly affect translation in HEK293T cells. Similar to observations in neuronal stimulation, YTHDF1 might be in a dormant state in HEK293T cells analogous to the resting state in neurons, while in Hela cells YTHDF1 may be constitutively activated. Importantly, YTHDF1 is highly expressed in Hela cells and seems to constitutively upregulate translation. However, the protein level of FMRP is much higher in HEK293T cells compared to HeLa. It is suspect that these differences underlie the observation that YTHDF1 does not noticeably promote translation in HEK293T cells, a property similar to WT mouse hippocampal neurons. Thus, experiments were performed to determine whether the regulatory role of FMRP on YTHDF1 could modulate the YTHDF1-mediated translation in different cellular systems. The relative protein levels of YTHDF1 and FMRP could dictate translation promotion function of YTHDF1.

Nascent protein synthesis assay was in control and short hairpin RNA (shRNA)-enabled stable knockdown cell lines. The results agreed with the previous report that loss of YTHDF1 led to a decrease of protein synthesis rate in Hela cells but a minor increase in HEK293T cells. A tethering reporter assay was performed in HEK293T and HeLa cells and quantified mRNA translation efficiency by dividing the protein level by the steady state mRNA level. The translation efficiency of YTHDF1 target RNA decreased in HeLa cells but increased in HEK293T cells upon YTHDF1 knockdown, which is consistent with the change in the general protein synthesis rate. These data support the hypothesis that YTHDF1 is in a dormant state in HEK293T cells but is constitutively activated in HeLa cells.

Experiments indicate that FMRP phosphorylation activates YTHDF1-mediated translation and FMRP dephosphorylation represses YTHDF1-mediated translation. FMRP phosphorylation was first inhibited in HeLa cells and protein level reduction was observed of two proteins encoded by known YTHDF1 target mRNAs, eEF4G and LRPAP1. Inhibition of FMRP phosphorylation decreases translation efficiency of YTHDF-tethered reporter mRNA in HeLa cells, and inhibition of FMRP dephosphorylation increases translation efficiency of YTHDF1-tethered reporter mRNA in HEK293T cells. Moreover, inhibition of FMRP phosphorylation led to decreased association between YTHDF1 and the 40S ribosome (presented by the proximity to RPS6), coinciding with an increased association between YTHDF1 and FMRP. Moreover, FMRP was knocked down in HEK293T cells. Condensation formation of YTHDF1 and RPS6 was quantified. Increased existence of YTHDF1 and RPS6 was observed in insoluble granules indicating that FMRP inhibits the granule formation of YTHDF1 and ribosomes.

To further investigate the exact stage of mRNA translation that YTHDF1-FMRP acts on, polysome fractions were separated from HeLa and HEK293T cells on a sucrose cushion. In HeLa cells, YTHDF1 notably co-exist with 40S ribosomal subunit, supporting its translation upregulation role. However, in addition to association with 40S ribosomal subunit, YTHDF1 was also found in heavy polysome fractions in HEK293T cells. FMRP phosphorylation in HeLa cells was inhibited to prevent YTHDF1-promoted translation. After p-FMRP inhibition, a redistribution of YTHDF1 to heavy polysomes was observed, similar to its native distribution in HEK293T cells.

To study individual effects of YTHDF1 in different polysome fractions, RNA was isolated from “monosome” and “polysome” fractions in HEK293T cells. Different translational events were studied. Consistent with the inactive state of YTHDF1 in HEK293T cells, YTHDF1 knockdown did not significantly alter translation initiations, while translation elongation of YTHDF1 targets was elevated; YTHDF1 in the polysome fraction in HEK293T cells appears to block translation elongation instead of promoting translation, which explains the increased protein synthesis rate after YTHDF1 knockdown in HEK293T cells. FMRP phosphorylation was inhibited in HeLa cells to induce polysome distribution of YTHDF1 in order to mimic that in HEK293T cells. Translation elongation of YTHDF1 target transcripts was significantly downregulated when FMRP phosphorylation was inhibited, whereas only a negligible reduction was observed for all m6A marked transcripts. These results indicate that unphosphorylated FMRP directs YTHDF1 distribution to heavy polysomes and these YTHDF1 proteins inhibit translation elongation, leading to inhibition of mRNA translation in its dormant state.

Although it is not intended that embodiments of this invention be limited by any particular mechanism. These experimental results suggest a mechanism of YTHDF1 activation by FMRP phosphorylation through simulation in neurons and other cells wherein non-phosphorylated FMRP recruits YTHDF1 and inhibits YTHDF1 association with the 40S ribosomal subunit. Phosphorylation of FMRP releases YTHDF1 to phase separate with the 40S ribosomal subunit and to promote translation of its target mRNA. The translation promotion function of YTHDF1 is thus regulated by its propensity to phase separate with different partner proteins.

### YTHDF1 is a Potential Target to Treat Fragile X Syndrome

Fragile X syndrome (FXS) is an example of a human disease exhibiting dysregulated translation. In FXS system, FMR1 expression is silenced by the hypermethylation of CGG expansion mutation in its promoter region. FMRP is primarily thought to be an inhibitor of mRNA translation events. The hippocampal slices from Fmr1 (negative)-KO mice incorporate 15-20% more [35S]methionine into nascent peptides when compared to wild-type control. Consistent with the translational repression role, FMRP was also reported to be associated with stalled polyribosomes. However, high-throughput studies of FMRP mRNA targets indicate that FMRP enhances the translation of its target mRNA. The association of FMRP-target mRNA with ribosome has been reported to reduce Fmr1-deficient neurons (Sawicka et al., 2019; Thomson et al., 2017). However, another study conducted in Drosophila oocytes indicates that FMRP maintains translation of a subset of long mRNAs (Greenblatt et al. 2018). These conflicting roles of FMRP on translation regulation may suggested involvement of multiple pathways in Fmr1-deficient systems. Activated mRNA translation through YTHDF1 with FMRP deficiency in cultured neurons and HEK293T cell line could potentially explain the hyperactive translation in FXS.

### Characterization of Salvianolic Acid C (SAC) as a Selective YTHDF1 Inhibitor

A fluorescence polarization (FP) based high throughput screening (HTS) assay was developed to search for inhibitors that may block the interaction between YTHDF1 and m6A-containing RNA. By screening a compound library, salvianolic acid C (SAC) (FIG. 1A), a well-known, water-soluble bioactive compound from the extractants of Salvia miltiorrhiza (Danshen), was found to be a potent hit. It was confirmed that SAC could effectively interfere binding of YTHDF1 to its substrate with an IC₅₀ value of about 1.4 μM in vitro (FIG. 1B).

To investigate the binding between SAC and YTHDF1, a series of qualitative and quantitative assays were conducted. The nuclear magnetic resonance (NMR) and Carr-Purcell-Meiboom-Gill (CPMG) experiments were performed. With a constant concentration of the compound, the signal detected would reduce when the concentration of YTHDF1 increased if there were interactions between the two. An obvious reduction of CPMG signal was observed under the conditions of 5 μM, 10 μM and 20 μM of YTHDF1, respectively, indicating direct binding between SAC and YTHDF1. The binding affinity between SAC and YTHDF1 was determined with an Isothermal Titration calorimetry (ITC) assay. The KD value of SAC against YTHDF1 was determined to be 6.3 μM, which once again confirmed direct binding between SAC and YTHDF1. Furthermore, the interaction strength between SAC and YTHDF1 was measured with a microscale thermophoresis (MST) experiment. The KD value of 5.3 μM determined by MST assay was consistent with that obtained from ITC experiments, further validating the binding between SAC and YTHDF1.

The mechanism of how SAC blocks YTHDF1 binding to m6A-containing RNA was explored. An AlphaScreen™ based substrate competition experiment was performed. If SAC is a substrate competitive inhibitor, the IC₅₀ value of the compound would alter with addition of unlabeled m6A-containing RNA. The IC₅₀ value of SAC against YTHDF1 increased from 1.7 μM to 46.9 μM with addition of unlabeled m6A-containing RNA from 0 nM to 400 nM. These results suggested that SAC is a substrate competitive inhibitor of YTHDF1. To investigate inhibition activity of SAC in cellular systems, a gradient of SAC concentrations was applied to HeLa cells, in which the translation of two proteins, eEF1G and LRPAP1 are known to be promoted by YTHDF1. The protein levels of eEF1G and LRPAP1 decreased in a dose-dependent manner with SAC treatment while mRNA levels remained unchanged, indicating SAC can inhibit YTHDF1 function in live cells.

Because the YTH domains of YTHDF1 and YTHDF2 share similar sequences, we next investigated the selectivity of SAC against YTHDF1 using the AlphaScreen™ assay. SAC interfered the interaction between YTHDF2 and m6A-containing RNA with the IC₅₀ value of 29.6 μM, which is about 20 times less effective than that against YTHDF, indicating that SAC possesses a high selectivity against YTHDF1 compared with YTHDF2. PRR5L expression upon SAC treatment was checked as PRR5L is known to be decayed through YTHDF2. The transcript level of PRR5L increased only upon YTHDF2 knockdown or treatment of 50 μM or higher concentrations of SAC. At 20 μM, which was used for most subsequent studies, YTHDF2 inhibition was not observed.

### Salvianolic Acid C (SAC) Rescues Neurodevelopmental Deficits in FXS Forebrain Organoids

Kang et al., 2021 report that loss of FMRP in human forebrain organoids could lead to reduced proliferation of neural progenitor cells, dysregulated neural differentiation, increased synapse formation and neuronal hyperexcitability, and a deficit in the production of GABAergic neurons. Given the effect of YTHDF1 modulation in FMRP deficient cells, experiments were performed to determine whether YTHDF1 inhibition could rescue developmentally altered phenotypes in FXS forebrain organoids. FXS forebrain organoids were developed from FXS patient-derived iPSCs (induced pluripotent stem cells). Both control and FXS forebrain organoids were treated with SAC at 20 μM from day 49 (D49) to 56 (D56).

To examine efficacy of SAC rescuing proliferation deficit of neural progenitor cells (NPCs) affected by the loss of FMRP, the cells were co-immunostained with Ki67, a proliferation marker, and NPC markers SOX2 or PAX6. Significant reductions of Ki67+ and SOX2+ NPC proliferation were observed in FXS forebrain organoids compared to control organoids. Notable increases of Ki67+ in proliferating NPCs in SAC-treated FXS forebrain organoids up to comparable levels as control forebrain organoids were observed. A slight increase in the proportion of Ki67+ NPCs in control forebrain organoids by SAC treatment was also observed.

After showing that SAC can rescue NPC proliferation in FXS forebrain organoids, the cell cycle kinetics with thymidine analogue EdU (5-ethynyl-2′-deoxyuridine) labeling was examined. Cell cycle exit was assessed by measuring the proportion of Ki67− and EdU+ cells among total EdU+ cells. The subset of Ki67− and EdU+ cells indicates that the cells exited cell cycle within 24 hours of labeling with EdU. Kang et al., 2021 report the number of Ki67− and EdU+ cells was markedly increased in D56 FXS forebrain organoids. This increase of Ki67− and EdU+ cells suggest an accelerated cell cycle exit in FXS organoids compared to the control forebrain organoids. The bath application of SAC for a week to organoid culture significantly reverted the phenotype of aberrantly accelerated cell cycle exit in FXS forebrain organoids. SAC treatment did not cause a change of cell cycle exit in control forebrain organoids in which system YTHDF1 was inactive.

To further delineate the effect of SAC on the neuronal differentiation defect caused by the loss of FMRP, the distribution of specific cell types (PAX6+ or CTIP2+ cells) were determined in bins which were equally divided through the entire span of neuroepithelium of forebrain organoids. The number of PAX6+ NPCs was significantly reduced in the ventricular zone (VZ)-like layer spanning lower Bins while it was increased in the cortical plate layer (higher Bins) of FXS forebrain organoids compared to control organoids. The alteration in PAX6+ NPC differentiation in FXS organoids was rescued after treatment with SAC. CTIP2+ cortical plate neurons dramatically increase in Bin2 and 3 in DMSO-treated FXS forebrain organoids compared to DMSO-treated control organoids, indicating the dysregulated neuronal differentiation and layer specification by FMRP loss. When the FXS organoids were treated with SAC, the dysregulated CTIP2+ neuronal differentiation and layer organization were partially rescued to a level comparable to control forebrain organoids. Importantly, SAC treatment in control forebrain organoids did not alter CTIP2+ neuronal differentiation and layer organization. The specific effect of SAC in NPC proliferation in FXS model is consistent with the notion that YTHDF1 is inactive in FMRP abundant systems.

These results indicate that the small molecule YTHDF1 inhibitor, SAC, could rescue the defects of decreased proliferation of neural progenitor cells, hastened cell cycle exit and altered neural differentiation in FXS organoids. Thus, dysregulated YTHDF1 RNP granule formation upon FMRP deficiency could lead to disease phenotypes in FXS. These results further indicate YTHDF1 as a viable target for treating FXS, opening up possibilities for future therapy development. The dysregulation of RNP granule equilibrium could guide drug target discovery in other human diseases showing alterations in RBPs.

YTHDF1 can Promote mRNA Translation in HeLa and HEK293T Cells

YTHDF1 can form RNP granules with small ribosomal subunit to promote target mRNA translation and that high levels of FMRP inhibits YTHDF1. In Hela cells, YTHDF1 is more abundant than FMRP and constitutionally promotes mRNA translation. In HEK293T cells, FMRP is more abundant than YTHDF1 with YTHDF1 hijacked away from ribosome containing RNP granules by FMRP to become inactive in promoting translation. FMRP phosphorylation or FMRP knockdown reverse the inhibition and activates the YTHDF1-mediated translation in HEK293T cells. With these results one can contemplate a molecular mechanism explaining the cell context dependent translation effect of YTHDF1.

However, these findings are not completely consistent with a recent report that all YTHDF proteins (YTHDF1, YTHDF2 and YTHDF3) are primarily engaged in synergistic regulation of mRNA decay without direct effects on translation (Zaccara et al., 2020). There were several points raised that argue against a distinct role of YTHDF1 in translation upregulation in even HeLa cells: 1) YTHDF1 and YTHDF2 share same protein partners for mRNA decay; 2) YTHDF1 and YTHDF2 share same mRNA targets and YTHDF1 knockdown does not affect mRNA translation; 3) YTHDF proteins share similar amino acid sequence and possess same functions on mRNA.

Experiments were performed to determine how relative levels of YTHDF1 and YTHDF2 and their interactions may alter granule dynamics and interactions with their corresponding protein partners and target mRNA. Most importantly, how triple knockdown of YTHDF proteins could lead to notable stabilization of cellular mRNAs.

YTHDF1 and YTHDF2 have Distinct Protein Partners

To study the protein partners of YTHDF1 and YTHDF2 under physiological conditions in HEK293T cells, protein co-immunoprecipitation (co-IP) was performed at 37° C. with antibodies against endogenous YTHDF1 and YTHDF2 followed by LC-MS/MS identification. Distinct protein partners were observed with large subsets of unique proteins identified for YTHDF1 and YTHDF2, respectively. YTHDF2-specific protein partners include CARPIN1. YTHDF1-specific protein partners include ribosomal protein RPS5. Differing from the report that YTHDF proteins primarily interact with CNOT proteins to mediate mRNA decay, unique high-confidence YTHDF1 protein partners were identified. With the analyses of co-IP datasets under physiological conditions and public datasets, the data suggests that YTHDF1 and YTHDF2 have distinct protein partners that are involved in different aspects of mRNA fate.

YTHDF1 Promotes Translation of its Target mRNA in Hela Cells

Reports suggesting that YTHDF1 does not promote translation in HeLa cells (Zaccara 2020) is inconsistent with tethering reporter assays in Hela cells and the activation of YTHDF1-mediated translation upon FMRP phosphorylation. Collectively, analysis using mRNA targets of YTHDF1 showed that YTHDF1 promotes the translation efficiency of its mRNA targets and does not affect the abundance of these targets. Experiments indicated distinct roles of YTHDF1 and YTHDF2 on their corresponding target transcripts. Given that multiple readers and diverse functional outcomes exist for m6A-modified transcripts, clearly defining mRNA targets of individual reader is important for the interpretation of their functions.

### YTHDF Proteins Differ in LLPS Granule Formation

After showing YTHDF proteins can bind to specific RNA targets and display distinct functions under native conditions, the molecular mechanism underlying their functional distinctions were explored. The YTHDF family proteins share similar sequences and structures, which were proposed as basis that they could be functionally redundant. Amino acid sequences of YTHDF proteins were analyzed. They differ mainly between residue 260 to residue 360, which is part of the prion-like domain (PLD) that contributes to the disorder rate of YTHDF proteins. After calculating amino acid similarities, it was identified that YTHDF1 and YTHDF3 are more similar with each other while YTHDF2 is more distinct to the other two. Moreover, the fine structures of purified YTHDF proteins were examined under electron microscope. YTHDF1 and YTHDF3 formed similar ring-like structures, while YTHDF2 formed fibril like structures. Elongated incubation of YTHDF2 promoted the formation of clear fibril like structure, highlighting the structural distinction of YTHDF2. Collectively, YTHDF proteins are different in amino acid sequence in PLD and form distinct structures in LLPS. These findings highlight the RNP granule formation as one mechanism to study individual functions of YTHDF proteins.

Different mRNA Targets of YTHDF1 and YTHDF2 in RNP Granules Using Phase-CLIP

Phase-CLIP was performed with antibodies against endogenous YTHDF1 and YTHDF2 in WT HEK293T cells. Results indicated that YTHDF1 and YTHDF2 bind similar subsets of mRNA using regular CLIP assay that dissociates RNP granules. When RNP granules were preserved under native conditions, distinct mRNA targets were observed between YTHDF1 and YTHDF2. Analysis of protein binding peaks on mRNA confirmed this conclusion. Using the regular CLIP assay YTHDF1 and YTHDF2 showed similar binding profiles; however, when preserved RNP granules were exposed o RNase treatment under more native conditions, the peak distribution of YTHDF1/2 proteins exhibited distinct patterns. The differences observed for YTHDF1 and YTHDF2 binding to mRNA indicate that heterogeneous RNA binding is likely to be a granule effect. Moreover, YTHDF1 peaks were shifted to the 3′ UTR and YTHDF2 peaks were shifted to 5′ UTR when comparing Phase-CLIP results versus CLIP results, further implicating that condensate formation not only affect transcriptome binding of YTHDF proteins but also binding sites on specific mRNA.

To validate the biological relevance of Phase-CLIP YTHDF1 RNA targets, function analysis was performed, and Ribo-seq in Fmr1 KO were examined in neural stem cells. Consistent with the translation promotion role of YTHDF1, YTHDF1 targets enriched in granules (“RNase+ unique”) exhibited the highest translation efficiency while the YTHDF1 targets in soluble form (“RNase-unique”) exhibited translation efficiency indistinguishable from non-targets. This suggests that knockout of Fmr1 would lead to YTHDF1 condensation with ribosome and upregulation of translation. Indeed, significant translation upregulation of LLPS-specific YTHDF1 targets were found in Fmr1 knockout cells, while the YTHDF1 targets identified without phase separation remained unchanged. These observations strongly support a model of YTHDF1 activation and indicate that methods to detect LLPS-specific RNA targets would be applicable to other systems.

YTHDF Triple Depletion Leads to Increased RNP Granule Formation and Universal Stabilization of mRNA

YTHDF proteins form an interconnected network in the cytosol to determine the fate of m6A-tagged mRNA in addition to their individual functions. Depletion of all three YTHDF proteins led to synergistic effect in RNA stabilization. To study the molecular mechanism of the RNA stabilization upon depletion of all YTHDF proteins, m6A “writer” METTL3 was knocked down in HeLa cells. Processing body (P-body) and G3BP1 condensate formation was investigated as these LLPS granules are main hubs for cytosolic RNA processing. Consistent with a role of m6A to facilitate RNP formation by recruiting YTHDF proteins, depletion of METTL3 or YTHDF1 drastically diminished both P-body and G3BP1 condensates. However, depletion of all three YTHDF proteins led to more P-body and G3BP1 condensate in HeLa cells. As P-body and G3BP1 condensates have been reported to shield RNA from decay, this increased formation of phase-separated granules might contribute to the stabilization of RNA observed upon YTHDF triple knockdown. Changes of m6A-tagged transcripts were studied after depletion of YTHDF proteins. Consistent with the change of P-body abundance and its RNA stabilization role, depletion of YTHDF1 led to shortened lifetime of P-body-enriched transcripts, while YTHDF triple knockdown led to stabilization of P-body-enriched transcripts. To further dissect P-body dependent and independent RNA stabilization effects, transcripts were grouped by states of m6A modification and P-body enrichment. YTHDF2 knockdown led to significant stabilization of m6A-modified transcripts in both P-body enriched and P-body depleted pools agreeing with its ability to recruit CCR4-NOT and promote RNA deadenylation, while this effect was not that significant with YTHDF1 knockdown. Importantly, the most significant stabilization of the entire transcriptome, independent of m6A modification, was observed after YTHDF triple knockdown, indicating the synergistic RNA decay by YTHDF proteins could be a result of increased P-body abundance in cells. This indicates that YTHDF proteins have distinct molecular structures and have distinct functions in regulating RNA processing. The synergistic effect in RNA stabilization could be a result of increased RNP granule formation to sequester RNA from decay, such as P-body.

### Phosphorylation of FMRP Regulates Translation Promotion Function of YTHDF1

Condensate formation is increasingly recognized as a functional process for the assembly of RNP granules with spatially segregated functions, enabling precise regulation for individual biological processes. YTHDF proteins, bearing intrinsic disordered domains (ILDs) in their amino acid sequence, have been shown to facilitate segregation of m6A-modified RNA into phase separated granules. YTHDF1 and YTHDF2 exhibit distinct ultrafine structures. While the m6A-binding domain YTH domain is conserved, the YTHDF proteins differ in their ILDs. The different ILDs most likely led to distinct protein interactomes of YTHDF proteins and their distinct functional outcomes.

YTHDF1 is reported to promote translation in cells by recruiting eIF3b and facilitate looping of actively translating RNA in Hela cells. However, YTHDF1 was also found to pose minimal effects on mRNA translation in HEK293T cells. Activity regulated YTHDF1 activation in neurons indicates an uncharacterized regulatory pathway of YTHDF1. Investigation of neurons and HEK293T cells in which YTHDF1 activity on translation is dormant revealed a role of FMRP and its phosphorylation. Unphosphorylated FMRP binds YTHDF1 and inhibits its translation upregulation function. Activity-regulated phosphorylation of FMRP reduces its interaction with YTHDF1 and releases it to associate with 40S ribosomal subunit, which leads to translation upregulation. This mechanism was confirmed in HeLa and HEK293T cells. When FMRP phosphorylation was reduced in HeLa cells reduced translation promotion through YTHDF1 was observed. When FMRP phosphorylation in HEK293T cells was increase, increased translation promotion through YTHDF1 was observed. The phosphorylation-dependent switch of FMRP regulates the compartmentalization of YTHDF1 and YTHDF1-bound RNA into translational silent and translational active RNP granules. This is an example of post-translational modifications (PTM) serving as phase separation switches for functional segregation.

### Small Molecule Inhibition of YTHDF1 Alleviates Development Defects in FXS

With the notion that FMRP inhibits YTHDF1-mediated translation, experiments were performed to determine whether hyper-activated translation in fragile X syndrome could be re-balanced with YTHDF1 inhibition. Targeting YTHDF1-mediated translation could alleviate defects in FXS. Salvianolic acid C (SAC) is identified as a non-covalent YTHDF1 inhibitor. Its effect in an FXS forebrain organoid model were evaluated. YTHDF1 inhibition effectively rescued the defects of decreased proliferation of neural progenitor cells, hastened cell cycle exit, and altered neural differentiation in FXS model. SAC treatment does not pose significant effects in control organoid models, further confirming that the rescued phenotypes are dependent on the YTHDF1 activation process in FMRP-deficient system. This result confirms YTHDF1 as a potential drug target in treating FXS and important roles of RNA m6A methylation on aberrant translation in FXS. The identification of dysregulated YTHDF1 RNP granule in FXS indicates uses of YTHDF1 inhibitors in other loss-of-function neuronal diseases or defects related to cytosol RNA processing and translation. As loss-of-function gene perturbations are usually challenging drug targets because of the difficulty to supply protein agonists, targeting RNP granule dynamics opens up various rescue or compensation possibilities.

1. A method of treating fragile X syndrome comprising administering an effective amount of a YTHDF inhibitor to a subject in need thereof.

2. The method of claim 1, wherein the YTHDF inhibitor is a selective small-molecule inhibitor of YTHDF1.

3. The method of claim 1, wherein the YTHDF inhibitor is (E)-3-(3,4-dihydroxyphenyl)-2-((3-(2-(3,4-dihydroxyphenyl)-7-hydroxybenzofuran-4-yl)acryloyl)oxy)propanoic acid [salvianolic acid C], derivatives, prodrugs, esters, or salts thereof.

4. The method of claim 1, wherein the YTHDF inhibitor is an antibody with an epitope on a YTHDF1 polypeptide.

5. The method of claim 1, wherein the YTHDF inhibitor is interfering shRNA or vector encoding interfering shRNA that targets YTHF1 mRNA or an antisense nucleobase polymer that targets YTHF1 mRNA.

6. A method of treating a developmental disorder comprising administering an effective amount of a YTHDF inhibitor to a subject in need thereof.

7. The method of claim 6, wherein the YTHDF inhibitor is a selective small-molecule inhibitor of YTHDF1.

8. The method of claim 6, wherein the YTHDF inhibitor is (E)-3-(3,4-dihydroxyphenyl)-2-((3-(2-(3,4-dihydroxyphenyl)-7-hydroxybenzofuran-4-yl)acryloyl)oxy)propanoic acid [salvianolic acid C], derivatives, prodrugs, esters, or salts thereof.

9. The method of claim 6, wherein the YTHDF inhibitor is an antibody with an epitope on a YTHDF1 polypeptide.

10. The method of claim 6, wherein the YTHDF inhibitor is interfering shRNA or vector encoding interfering shRNA that targets YTHF1 mRNA or an antisense nucleobase polymer that targets YTHF1 mRNA.

11. The method of claim 6 wherein the developmental disorder is a poor motor coordination, prominent ears, a long face, flat feet, hyperextensible finger joints, double-jointed thumbs, an infant with weak sucking and/or frequent regurgitation.

12. A method treating a cognitive disorder comprising administering an effective amount of a YTHDF inhibitor to a subject in need thereof.

13. The method of claim 12, wherein the YTHDF inhibitor is a selective small-molecule inhibitor of YTHDF1.

14. The method of claim 12, wherein the YTHDF inhibitor is (E)-3-(3,4-dihydroxyphenyl)-2-((3-(2-(3,4-dihydroxyphenyl)-7-hydroxybenzofuran-4-yl)acryloyl)oxy)propanoic acid [salvianolic acid C], derivatives, prodrugs, esters, or salts thereof.

15. The method of claim 12, wherein the YTHDF inhibitor is an antibody with an epitope on a YTHDF1 polypeptide.

16. The method of claim 12, wherein the YTHDF inhibitor is interfering shRNA or vector encoding interfering shRNA that targets YTHF1 mRNA or an antisense nucleobase polymer that targets YTHF1 mRNA.

17. The method of claim 12, wherein the cognitive disorder is an autism spectrum disorder, attention-deficit/hyperactivity disorder (ADHD), speech impediment, hyperactivity, seizure, and/or hypotonic state.
